(19) United States
(12) Patent Application Publication
(10) Pub. No.: US 2018/0256766 A1
(43) Pub. Date: Sep. 13, 2018

(54) BRACKET ASSEMBLY

(71) Applicant: Avalign Technologies, Inc., Bannockburn, IL (US)

(72) Inventors: Warren Scott Gareiss, Warsaw, IN (US); Kristen Nichole Schackmann, Greenwood, IN (US); Ryan Michael Ratkowski, Fort Wayne, IN (US)

(21) Appl. No.: 15/918,358

(22) Filed: Mar. 12, 2018

Related U.S. Application Data
(60) Provisional application No. 62/470,398, filed on Mar. 13, 2017.

(51) Int. Cl. A61L 2/26; F16B 2/22; A61B 50/33; A61L 2/07

(52) U.S. Cl. CPC A61L 2/26; F16B 2/22; A61B 2090/0813; A61L 2/07; A61B 50/33

(57) ABSTRACT

Some embodiments of the invention may provide a bracket assembly for selectively receiving a stem portion of a medical device therein. The bracket assembly may be movably or permanently disposed within a case or tray assembly. The case or tray assembly may then be placed within a sterilization chamber such as an autoclave to sterilize the medical device. The bracket assembly includes a main body coupled with a gripping element for selectively clamping or otherwise holding the stem portion of the medical device therein in response to pressing the medical device into the gripping element.

### BACKGROUND

In some instances, it may be desirable to provide a sterile medical device such as a medical device or a medical implant for reuse in a medical procedure. One way of sterilizing the medical device is to place the medical device into a case or tray assembly and thereafter place the case or tray assembly into a sterilization device such as an autoclave.

### SUMMARY OF THE INVENTION

Some embodiments of the invention may provide a bracket assembly for selectively receiving a portion of a medical device therein. The bracket assembly may be movably or permanently disposed within a case or tray assembly. The tray assembly may then be placed within a sterilization chamber such as an autoclave to sterilize the medical device.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a perspective view of an exemplary tray assembly with multiple exemplary bracket assemblies disposed therein, with two bracket assemblies receiving a portion of an exemplary medical device;

FIG. 2 depicts a perspective view of an exemplary main body of the bracket assembly of FIG. 1;

FIG. 3 depicts a perspective view of an exemplary gripping element of the bracket assembly of FIG. 1;

FIG. 4 depicts a perspective view of the bracket assembly of FIG. 1;

FIG. 5 depicts a right side elevational view of the bracket assembly of FIG. 4;

FIG. 6 depicts a top plan view of the bracket assembly of FIG. 4;

FIG. 7 depicts a front elevational view of the bracket assembly of FIG. 4 with the gripping element in a default position and the medical device being lowered into the gripping element;

FIG. 8 depicts a front elevational view of the bracket assembly of FIG. 4 with the gripping element transitioning from the default position to a first gripping position, with the medical device abutting the gripping element;

FIG. 9 depicts a front elevational view of the bracket assembly of FIG. 4 with the gripping element in the first gripping position and the medical device secured therein;

FIG. 10 depicts a front perspective view of the bracket assembly of FIG. 4 with the gripping element in the second gripping position and the medical device secured therein;

FIG. 11 depicts a front elevational view of the gripping element of FIG. 3 with an exemplary support element shown in the default position in solid lines and shown in the second gripping position in dashed lines; and

FIG. 12 depicts a method of using the bracket assembly and tray of FIG. 4.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Tray Assembly

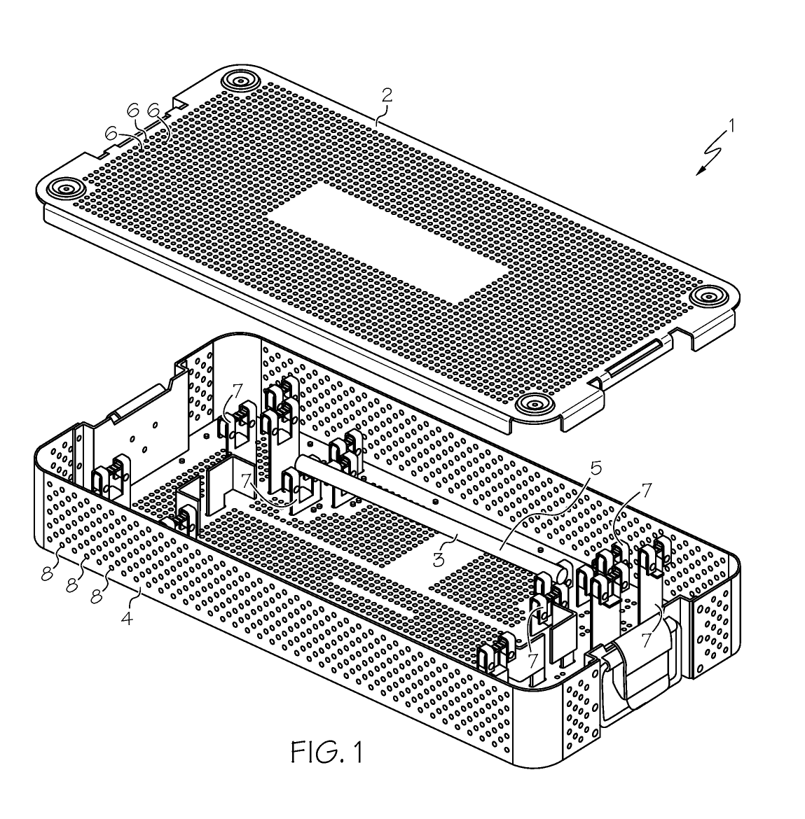

As shown in FIG. 1, some aspects of the present invention relate to a tray assembly (1) for use in sterilizing a medical device (3). Medical device (3) generally includes at least one stem portion (5). Tray assembly (1) includes a lid (2) defining a plurality of lid openings (6) and a base (4) defining a plurality of base openings (8) therein. Base (4) is configured to receive a bracket assembly (7) of the present invention therein. Bracket assembly (7) is configured to selectively receive stem portion (5) of medical device (3) to releasably hold medical device (3) firmly within tray assembly (1).

Lid (2) may be placed on base (40) and used as a top portion of tray assembly (1). Lid (2) may include bumpers (not shown) or other portions aligned with bracket assembly (7) to aid in holding medical device (3) in bracket assembly (7).

Tray assembly (1) may thereafter be moved or placed into a sterilization chamber without disturbing or agitating medical device (3) from a secure connection with base (4) via bracket assembly (7). As needed, medical device (3) may be removed from tray assembly (1) by releasing stem portion (5) from bracket assembly (7). As shown in FIG. 1, multiple bracket assemblies (7) may be used to hold a single medical device (3), with some embodiments of the present invention comprising bracket assemblies (7) disposed at both the first end and second end of medical device (3).

II. Exemplary Bracket Assembly

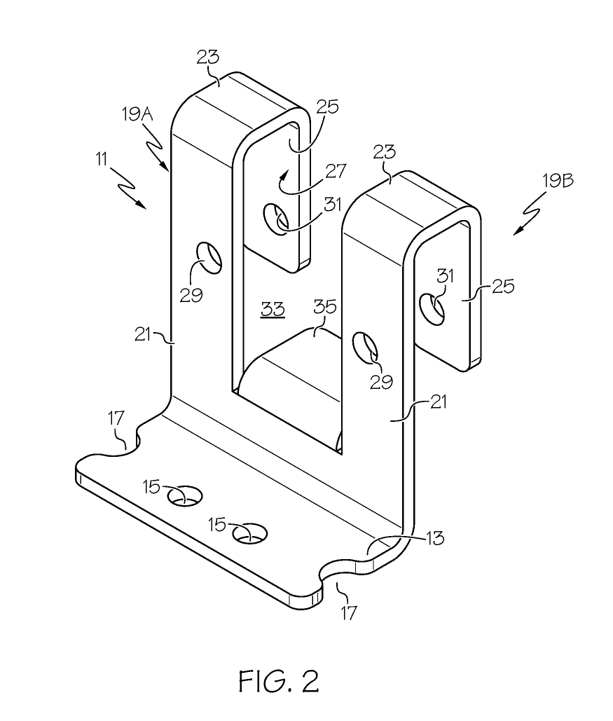

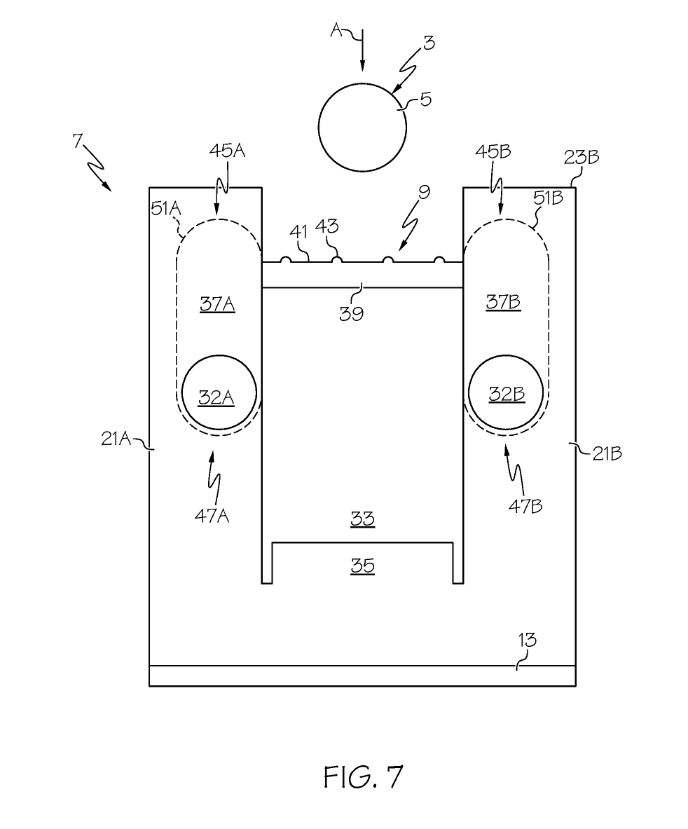

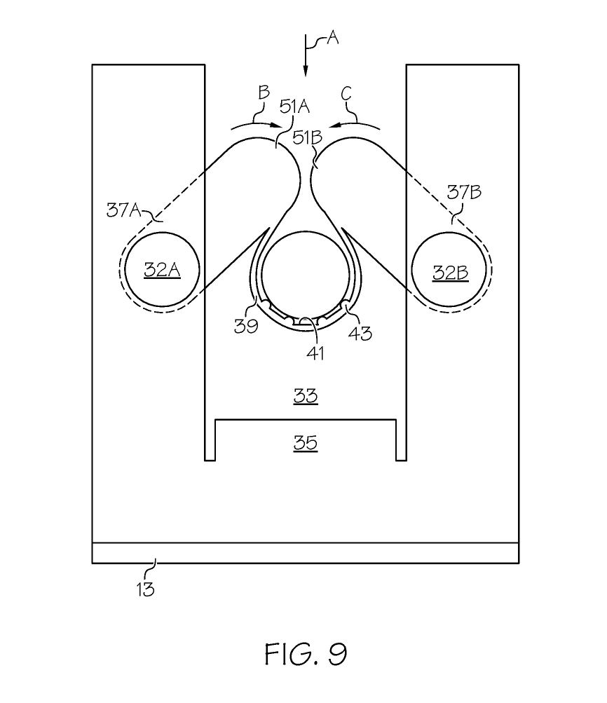

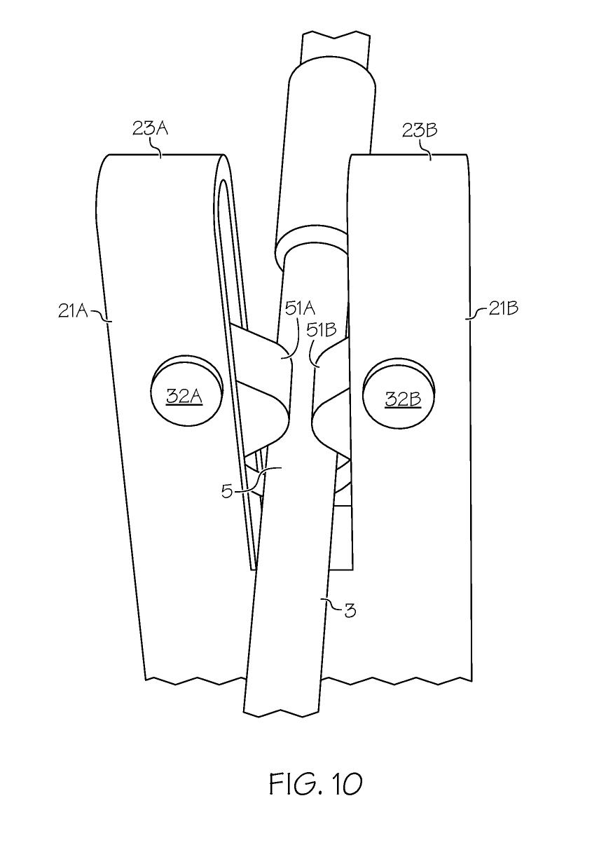

As shown in FIGS. 2-11, bracket assembly (7) includes a gripping element (9) coupled with a main body (11). Gripping element (9) is configured to move between a default position (FIG. 7) to either a first gripping position (FIG. 9) or a second gripping position (FIG. 10), depending upon the diameter of stem portion (5) of the particular medical device (3) pressed into gripping element (9). Gripping element (9) moves into the first gripping position (FIG. 9) for larger diameter stem portions (5) and moves into the second gripping position (FIG. 10) for smaller diameter stem portion (5).

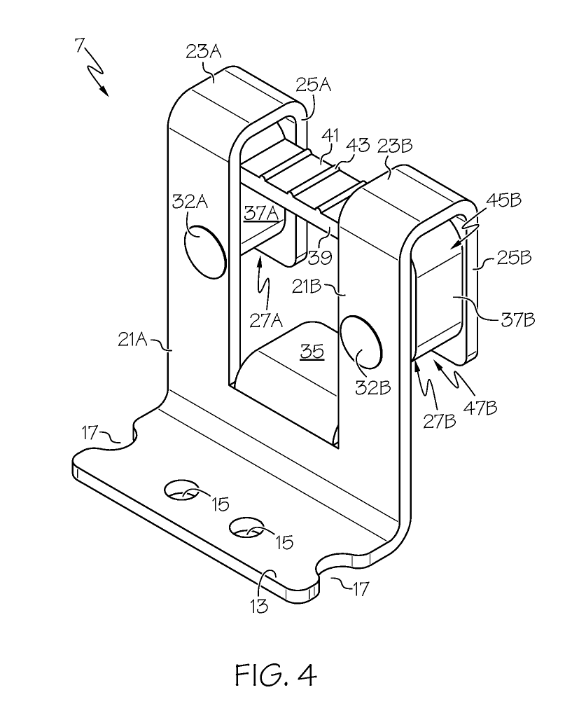

As shown in FIG. 2, some versions of main body (11) include a base flange (13) which defines two attachment openings (15) and two notches (17). Notches (17) aid in the positioning of bracket assembly (7) within base (4) by providing a visually distinguishing feature for generally similar components. Notches (17) may be formed in various shapes, sizes, and locations on bracket assembly (7) and may vary in number. A pair of arm elements (19A, 19B) extend upwardly away from base flange (13) in a generally perpendicular direction. Each arm element (19A, 19B) includes a first segment (21), a second segment (23), and a third segment (25). First segment (21), second segment (23), and third segment (25) cooperate to define a U-shaped channel (27) therebetween. As shown in FIG. 4, arm element (19A) defines a first U-shaped channel (27A) and arm element (19B) defines a second U-shaped channel (27B).

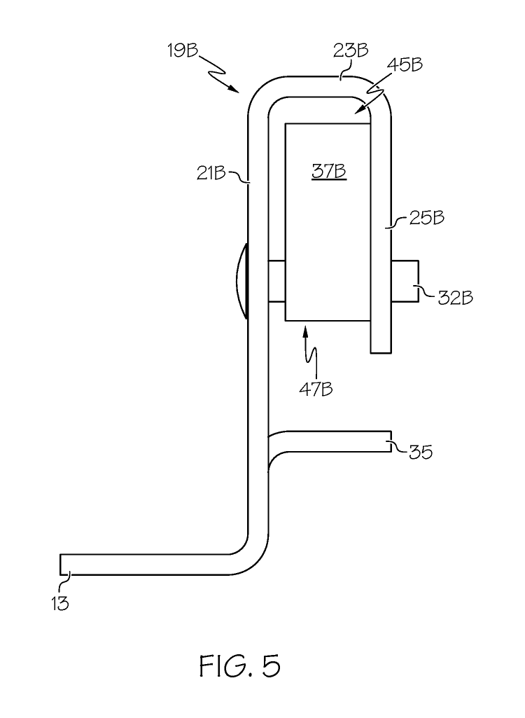

As shown in FIG. 5, in some versions of main body (11), first segment (21) extends perpendicularly from base flange (13). In some versions of main body (11), second segment (23) extends perpendicularly from first segment (21). In some versions of main body (11), third segment extends perpendicularly from second segment (23).

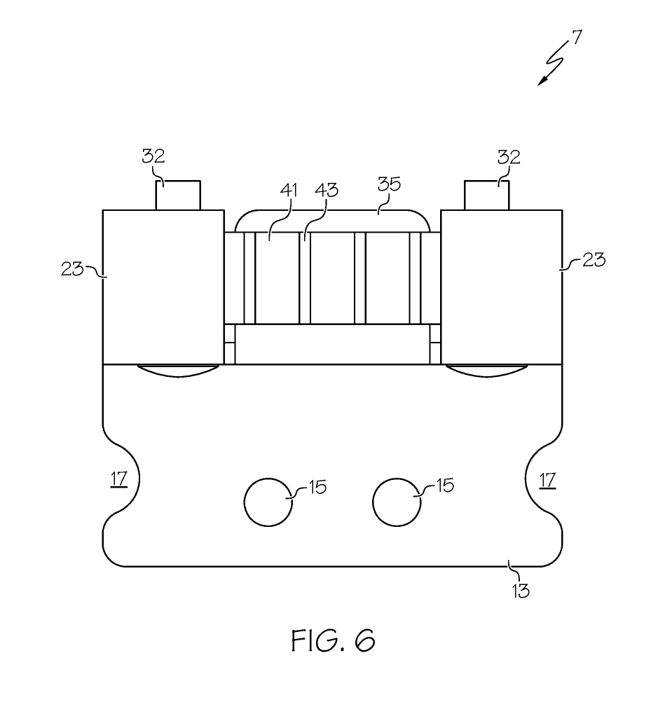

First segment (21) defines an opening (29) and third segment (25) defines an opening (31). As shown in FIGS. 5 and 6, opening (29) and opening (31) are generally aligned such that a generally linear bolt or fastener (32) can rest within both opening (29) and opening (31) simultaneously. An upper flange (35) is disposed between the pair of arm elements (19A, 19B) and is generally parallel to base flange (13). Upper flange (35) acts as a stop for the insertion of medical device (3) as well as a support structure. Upper flange (35) and arm elements (19A, 19B) define an inner channel (33) therebetween.

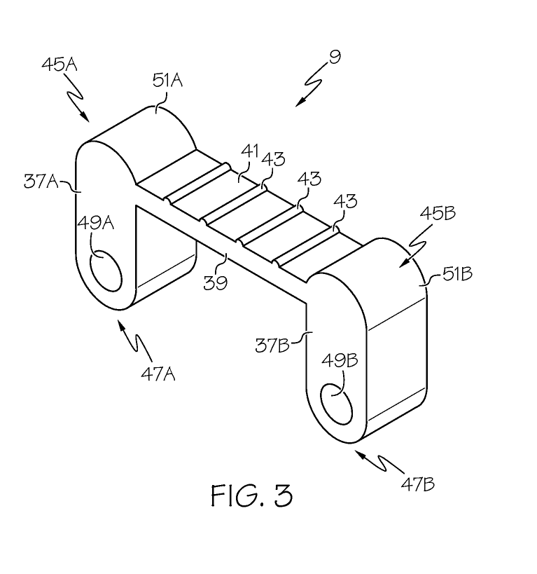

Referring now to FIG. 3, gripping element (9) includes a pair of support elements (37A, 37B) and a bridge element (39) extending therebetween. Bridge element (39) includes an upper surface (41) with a plurality of ribs (43) extending outwardly away therefrom. Each support element (37A, 37B) extends from a top portion (45) to a bottom portion (47) and defines a channel (49) proximate bottom portion (47). A protuberance (51) is disposed at top portion (45) of each support element (37A, 37B), with each protuberance (51) extending vertically upwards from upper surface (41) of bridge element (39). The height of each protuberance (51) is configured to not abut the other protuberance (51) when support elements (37A, 37B) are extending towards one another in either the first gripping position shown in FIG. 9 or the second gripping position shown in FIG. 10.

As shown in FIGS. 4-6, gripping element (9) is configured to be rotatably connected with main body (11). To facilitate the connection, each support element (37A, 37B) of gripping element (9) is positioned at least partially within corresponding U-shaped channel (27A, 27B) of main body (11). With reference to FIG. 4, support element (37A) is configured to rotate at least partially within U-shaped channel (27A) and support element (37B) is configured to rotate at least partially within U-shaped channel (27B). The rotating nature of support elements (37A, 37B) provide support for applying clamping forces onto step portion (5) when gripping element (9) is in either the first gripping position (FIG. 9) or the second gripping position (FIG. 10). Channels (49) defined by gripping element (9) are aligned with first openings (29) and second openings (31) defined by each arm element (19) to allow fasteners (32) to connect gripping element (9) with main body (11). Specifically, as shown in FIGS. 2-4, first opening (29A) of first segment (21A) is aligned with channel (49A) of support element (37A) and second opening (31A) of third segment (25A). Similarly, first opening (29B) of first segment (21B) is aligned with channel (49B) of support element (37B) and second opening (31B) of third segment (25B). Once first openings (29), second openings (31), and channels (49) are aligned, fasteners (32) may be extended therethrough to connect gripping element (9) to main body (11) and form bracket assembly (7).

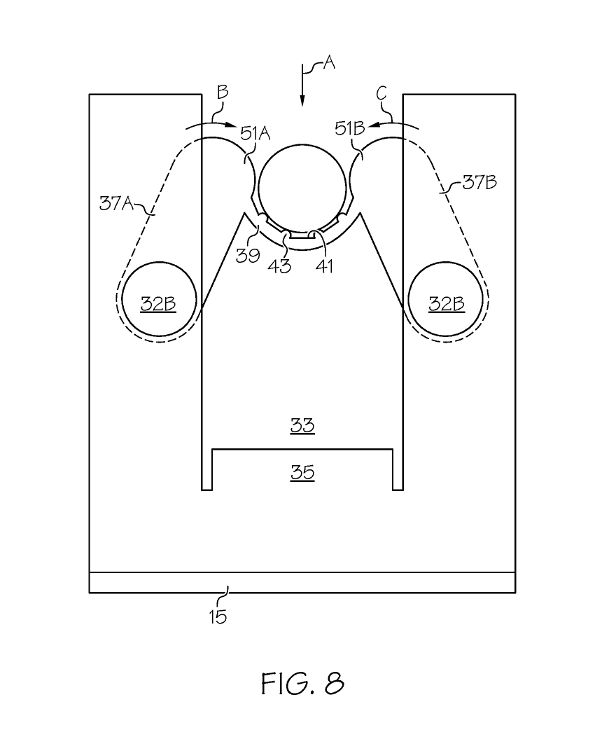

As shown in FIGS. 7-11, bracket assembly (7) is configured to move between a default position (FIG. 7) and either the first gripping position (FIG. 9) or the second gripping position (FIG. 10), depending on the diameter of stem portion (5) as stem portion (5) of medical device (3) is pressed into upper surface (41) and ribs (43) of bridge element (39). FIG. 7 depicts gripping element (9) in the default position, with stem portion (5) of medical device (3) approaching bridge element (39) in the direction of Arrow A. FIG. 8 depict gripping element (9) in a transition between the default position (FIG. 7) and the first gripping position (FIG. 9) or the second gripping position (FIG. 10), with stem portion (5) abutting and pressing upon upper surface (41) and ribs (43) of bridge element (39). The abutment and pressing into bridge element (39) by stem portion (5) begins to pivot each support element (37A, 37B) towards one another about their respective fasteners (32A, 32B). Protuberance (51A) of support element (37A) pivots in the direction of Arrow B and protuberance (51B) of support element (37B) pivots in the direction of Arrow C. The pivoting allows bridge element (39) to bend towards upper flange (35) with the apex of the bend in the general center of bridge element (39). Thus, in response to pressing stem portion (5) into gripping element (9) and wrapping gripping element (9) around stem portion (5) of medical device (3) to secure medical device (3) to bracket assembly (7), an apex is formed in bridge element (39) of gripping element (9).

FIG. 9 depicts gripping element (9) in the first gripping position, with stem portion (5) locked into place between protuberances (51A, 51B) and bridge element (39). Stem portion (5) is locked due to the rotation of protuberance (51A) in the direction of Arrow B and the rotation of protuberance (51B) in the direction of Arrow C. Bridge element (39) envelops most of stem portion (5) with ribs (43) abutting the surface of stem portion (5). As gripping element moves into the first gripping position, gripping element (9) is held in the first gripping position through a bending bias of bridge element (39) in a circular shape around stem portion (5).

FIG. 10 depicts gripping element (9) in the second gripping position. In the first gripping position, top portion (45) of each support element (37A, 37B) is generally directed upwards relative to stem portion (5) of medical device (3). In the second gripping position, top portion (45) of each support element (37A, 37B) is generally directed downwards relative to stem portion (5) of medical device (3). In both first and second gripping positions, support elements (37A, 37B) break over center to clamp stem portion (5) of medical device (3) between support elements (37A, 37B) with bridge element (39).

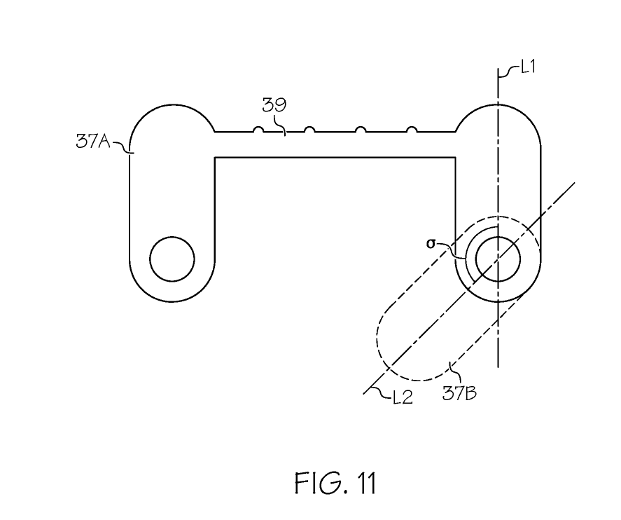

FIG. 11 depicts support element (37B) extending along a first line (L1) when gripping element (9) is the default position. Shown in dashed lines, when gripping element (9) is in the second gripping position, support element (37B) extends along a second line (L2). First line (L1) and second line (L2) extend at an angle (0) with respect to each other. In some versions of gripping element (9), angle (θ) is generally greater than 90 degrees and less than 180 degrees. In some versions of gripping element (9), angle (θ) is generally between 115 degrees and 155 degrees. Support element (37A) extends generally along the same angles as depicted in FIG. 11 with support element (37B). Thus, support elements (37A, 37B) move generally between 115 and 155 degrees between the default position and the second gripping position.

Ribs (43) provide spacing between stem portion (5) and upper surface (41) of bridge element (39) to allow for steam or other cleaning vapors or liquids to sterilize stem portion (5). The height of protuberances (51A, 51B) are configured to allow bridge element (39) to break over center without each protuberance (51A, 51B) abutting one another, while also allowing for a range of different stem portion (5) diameters or cross-sectional shape or size to be locked into bracket assembly (7). The length of bridge element (39) and the distance between each arm element (19A, 19B) provide for a universal fit for stem portions (5) of varying cross-sectional shape and size.

III. Method of Using Bracket Assembly

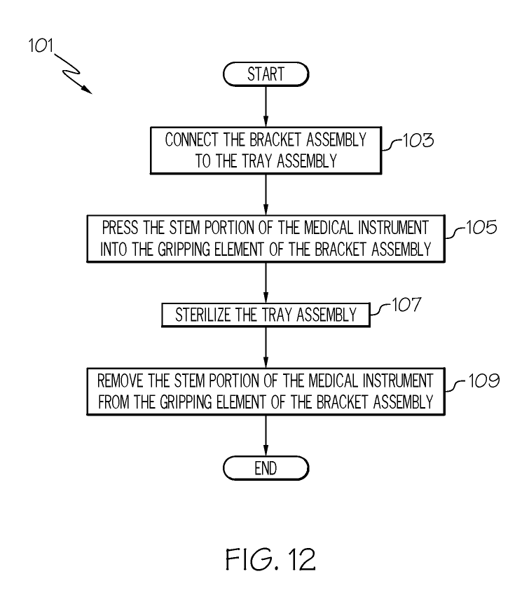

FIG. 12 depicts a method (101) of using bracket assembly (7). Method (101) begins with a step (103), whereby bracket assembly (7) is connected to base (4) of tray assembly (1). The connection may be provided through fasteners (not shown) extending through attachment openings (15) of main body (11) into base (4) of tray assembly (1). As shown in FIG. 10, multiple bracket assemblies (7) may be connected to base (4) of tray assembly (1) as needed to support multiple medical devices (3) or to support multiple portions of the same medical device (3). Once bracket assembly (7) is connected to base (4) of tray assembly (1) as desired, step (103) moves to a step (105).

In step (105), stem portion (5) of medical device (3) is manually pressed against bridge element (39) of bracket assembly (7). This results in gripping element (9) rotating from the default position (FIG. 7) to the locked or gripping position (FIG. 9). Bridge element (39) and protuberances (51) envelope stem portion (5) to lock medical device (3) within bracket assembly (7). After stem portion (5) of medical device (3) is locked within bracket assembly (7), step (105) proceeds to a step (107).

In step (107), tray assembly (1) containing medical device (3) locked within bracket assembly (7) is sterilized using any common mechanism know in the art. In some embodiments, tray assembly (1) is disposed in an autoclave where steam is used sterilize medical device (3) through openings (6) of lid (2) and openings (8) of base (4). Inasmuch as stem portion (5) is held apart from direct contact with upper surface (41) of bridge element (39) by ribs (43), steam penetrates around the periphery of stem portion (5) even while medical device (3) is locked within bracket assembly (7). After medical device (3) is sufficiently sterilized, step (107) proceeds to a step (109).

In step (109), medical device (3) is manually lifted outwardly away from bracket assembly (7) in the opposite direction from Arrow A in FIG. 7-9. The lifting of stem portion (5) away from bridge element (39) acts to abut protuberances (51A, 51B), moving protuberances (51A, 51B) in the opposite direction from Arrows B and C in FIGS. 7-9. The movement of protuberances (51A, 51B) begins to rotate support elements (37A, 37B), which further facilitates the release of medical device (3) from bracket assembly (7). The biased nature of bridge element (39) in the locked position acts to further the unlocking of gripping element (9) by way of releasing the bend in bridge element (39) once support elements (37A, 37B) begin to move back towards the default position. After medical device (3) is released from bracket assembly (7), method (101) proceeds to terminate.

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A bracket assembly comprising: (a) a main body comprising: (i) a first arm element, and (ii) a second arm element; and (b) a gripping element comprising: (i) a first support element, wherein the first support element is rotatably coupled with the first arm, (ii) a second support element, wherein the second support element is rotatably coupled with the second arm, and (iii) a bridge element, wherein the bridge element is disposed between the first arm element and the second arm element.

### EXAMPLE 2

The bracket assembly of Example 1 or any of the subsequent Examples, wherein the main body comprises a base flange configured to be coupled with a tray for use in sterilizing a medical device.

### EXAMPLE 3

The bracket assembly of any of the previous or subsequent Examples, wherein the base flange defines at least two attachment openings.

### EXAMPLE 4

The bracket assembly of any of the previous or subsequent Examples, wherein the base flange defines at least two notches.

### EXAMPLE 5

The bracket assembly of any of the previous or subsequent Examples, wherein the first arm comprises a first segment, wherein the first segment extends perpendicularly from the base flange.

### EXAMPLE 6

The bracket assembly of any of the previous or subsequent Examples, wherein the first arm comprises a second segment, wherein the second segment extends perpendicularly from the first segment.

### EXAMPLE 7

The bracket assembly of any of the previous or subsequent Examples, wherein the first arm comprises a third segment, wherein the third segment extends perpendicularly from the second segment.

### EXAMPLE 8

The bracket assembly of any of the previous or subsequent Examples, wherein the first segment defines a first opening, wherein the third segment defines a second opening.

### EXAMPLE 9

The bracket assembly of any of the previous or subsequent Examples, further comprising a fastener, wherein the fastener extends through the first opening, the first support element, and the second opening to rotatably couple the first support element with the first arm.

### EXAMPLE 10

The bracket assembly of any of the previous or subsequent Examples, wherein the gripping element is configured to rotate from a default position to a gripping position to secure a stem of a medical device therein.

### EXAMPLE 11

The bracket assembly of any of the previous or subsequent Examples, wherein the first support element extends along a first line in the default position, wherein the first support element extends along a second line in the gripping position, wherein the first line and the second line are oriented at an angle, wherein the angle is greater than 90 degrees and less than 180 degrees.

### EXAMPLE 12

The bracket assembly of any of the previous or subsequent Examples, wherein the angle is between 115 degrees and 155 degrees.

### EXAMPLE 13

The bracket assembly of any of the previous or subsequent Examples, wherein the first support element and the second support element do not abut in the default position, wherein the first support element and the second support element do not abut in the gripping position.

### EXAMPLE 14

The bracket assembly of any of the previous or subsequent Examples, wherein the main body comprises an upper flange disposed between the first arm and the second arm, wherein the bridge moves toward the upper flange when the gripping element rotates from the default position to the gripping position.

### EXAMPLE 15

The bracket assembly of any of the previous or subsequent Examples, wherein the main body comprises a base flange configured to be coupled with a tray for use in sterilizing a medical device, wherein the base flange is parallel with the upper flange.

### EXAMPLE 16

The bracket assembly of any of the previous Examples, wherein the gripping element is configured to move between a default position and a gripping position, wherein the gripping element is configured to form an apex in the gripping position.

### EXAMPLE 17

A device comprising: (a) a tray assembly comprising: (i) a lid, wherein the lid defines a plurality of lid openings therein, and (ii) a base, wherein the base defines a plurality of base openings therein; (b) a bracket assembly, wherein the bracket assembly is configured to be removably secured to the base of the tray assembly, the bracket assembly comprising: (i) a main body comprising: (A) a first arm element, wherein the first arm element defines a first U-shaped channel, and (B) a second arm element, wherein the second arm element defines a second U-shaped channel; and (ii) a gripping element comprising: (A) a first support element, wherein the first support element is rotatably coupled with the first arm, wherein the first support element is configured to rotate at least partially within the first U-shaped channel, (B) a second support element, wherein the second support element is rotatably coupled with the second arm, wherein the second support element is configured to rotate at least partially within the second U-shaped channel, and (C) a bridge element, wherein the bridge element extends between the first support element and the second support element.

### EXAMPLE 18

A method of sterilizing a medical device comprising: (a) connecting a bracket assembly to a tray; (b) pressing a medical device into the bracket assembly; (c) sterilizing the tray and the medical device held therein; and (d) removing the sterilized medical device from the bracket assembly.

### EXAMPLE 19

The method of Examples 18 or 20, further comprising, in response to pressing the medical device into the bracket assembly, wrapping a gripping element around the medical device to secure the medical device to the bracket assembly.

### EXAMPLE 20

The method of any of the previous Examples, in response to wrapping the gripping element around the medical device to secure the medical device to the bracket assembly, forming an apex in a bridge element of the gripping element.

V. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. A bracket assembly comprising:
(a) a main body comprising:
(ii) a first arm element, and
(ii) a second arm element; and
(b) a gripping element comprising:
(i) a first support element, wherein the first support element is rotatably coupled with the first arm,
(ii) a second support element, wherein the second support element is rotatably coupled with the second arm, and
(iii) a bridge element, wherein the bridge element is disposed between the first arm element and the second arm element.

2. The bracket assembly of claim 1, wherein the main body comprises a base flange configured to be coupled with a tray assembly for use in sterilizing a medical device.

3. The bracket assembly of claim 2, wherein the base flange defines at least two attachment openings.

4. The bracket assembly of claim 3, wherein the base flange defines a notch.

5. The bracket assembly of claim 2, wherein the first arm comprises a first segment, wherein the first segment extends perpendicularly from the base flange.

6. The bracket assembly of claim 5, wherein the first arm comprises a second segment, wherein the second segment extends perpendicularly from the first segment.

7. The bracket assembly of claim 6, wherein the first arm comprises a third segment, wherein the third segment extends perpendicularly from the second segment.

8. The bracket assembly of claim 7, wherein the first segment defines a first opening, wherein the third segment defines a second opening.

9. The bracket assembly of claim 8, further comprising a fastener, wherein the fastener extends through the first opening, the first support element, and the second opening to rotatably couple the first support element with the first arm.

10. The bracket assembly of claim 1, wherein the gripping element is configured to rotate from a default position to a gripping position to secure a stem of a medical device therein.

11. The bracket assembly of claim 10, wherein the first support element extends along a first line in the default position, wherein the first support element extends along a second line in the gripping position, wherein the first line and the second line are oriented at an angle, wherein the angle is greater than 90 degrees and less than 180 degrees.

12. The bracket assembly of claim 10, wherein the angle is between 115 degrees and 155 degrees.

13. The bracket assembly of claim 10, wherein the first support element and the second support element do not abut in the default position, wherein the first support element and the second support element do not abut in the gripping position.

14. The bracket assembly of claim 10, wherein the main body comprises an upper flange disposed between the first arm and the second arm, wherein the bridge element moves toward the upper flange when the gripping element rotates from the default position to the gripping position.

15. The bracket assembly of claim 14, wherein the main body comprises a base flange configured to be coupled with a tray for use in sterilizing a medical device, wherein the base flange is parallel with the upper flange.

16. The bracket assembly of claim 1, wherein the gripping element is configured to move between a default position and a gripping position, wherein the gripping element is configured to form an apex in the gripping position.

17. A device comprising:
(a) a tray assembly comprising:
(i) a base, wherein the base defines a plurality of base openings therein;
(b) a bracket assembly, wherein the bracket assembly is configured to be removably secured to the base of the tray assembly, the bracket assembly comprising:
(i) a main body comprising:
(A) a first arm element, wherein the first arm element defines a first U-shaped channel, and
(B) a second arm element, wherein the second arm element defines a second U-shaped channel; and
(ii) a gripping element comprising:
(A) a first support element, wherein the first support element is rotatably coupled with the first arm, wherein the first support element is configured to rotate at least partially within the first U-shaped channel,
(B) a second support element, wherein the second support element is rotatably coupled with the second arm, wherein the second support element is configured to rotate at least partially within the second U-shaped channel, and
(C) a bridge element, wherein the bridge element extends between the first support element and the second support element.

18. A method of sterilizing a medical device comprising:
(a) connecting a bracket assembly to a base of a tray assembly;
(b) pressing a medical device into the bracket assembly;
(c) sterilizing the tray assembly and the medical device held therein; and
(d) removing the sterilized medical device from the bracket assembly.

19. The method of claim 16, further comprising, in response to pressing the medical device into the bracket assembly, wrapping a gripping element around the medical device to secure the medical device to the bracket assembly.

20. The method of claim 17, in response to wrapping the gripping element around the medical device to secure the medical device to the bracket assembly, forming an apex in a bridge element of the gripping element.
